(19) United States
(12) Patent Application Publication
(10) Pub. No.: US 2003/0109495 A1
(43) Pub. Date: Jun. 12, 2003

(54) Nutraceutical solutions comprising dimethyl sulfoxide

(76) Inventor: Louis Frank Kretschmer, Olympia, WA (US)

Correspondence Address: Louis Frank Kretschmer M.D., 405A Black Hills LN S.W., Olympia, WA, 98502, US

(21) Appl. No.: 10/226,528

(22) Filed: Aug. 23, 2002

Related U.S. Application Data
(60) Provisional application No. 60/315,290, filed on Aug. 27, 2001.

(51) Int. Cl. A61K 31/7008; A61K 31/255

(52) U.S. Cl. 514/62; 514/517

(57) ABSTRACT

A unique topical treatment for diseases of joints and soft tissues with a proven scientific basis. The carrying agent is DMSO or (dimethyl sulfoxide) in purified form. This has ability to carry molecules of molecular weight 1000 Daltons or less through the skin. DMSO is also a proven anti-inflammatory for the treatment of joint and soft tissue diseases. By adjusting the acidity one can prepare a unique stable topical gel containing 5% glucosamine (molecular weight 200 Daltons) for the treatment of joint and ligament diseases. By adding 5% MSM we include a powerful anti-oxidant of sufficient low molecular weight to also be delivered through the skin. No other topical application presently available for the treatment of joint and soft tissue disorders can make such claims with scientific fact. No other topical can claim glucosamine, MSM, an anti-inflammatory and anti-oxidant in sufficient concentrations through the skin to have a beneficial affect on joints and soft tissues.

### FIELD OF INVENTION

This invention involves the topical application of nutraceuticals with dimethyl sulfoxide (DMSO) as a carrying agent.

### BACKGROUND OF THE INVENTION

DMSO was founded by my close friend Dr. Stanley Jacob at the University of Oregon Medical School which I attended. Its unique agent for low molecular weight compounds is unparalleled. Now we have medical grade DMSO that does not posses the unpleasant garlic taste and smell of previous impure preparations.

U.S. Pat. No. 3,549,720 as well as personal communication with Dr. Jacob confirms nutraceuticals (not pharmaceuticals) effectively treat tissue damage, arthritis pain, muscle pain.

U.S. Pat. No. 3,711,606 as well as my work and that of Dr. Jacob confirms 50% DMSO in gel and lotion form to penetrate all levels of the skin.

Compositions in the past have included large molecular weight medicines which are not readily carried across the skin.

Furthermore those medicines of the category Cox I inhibitors or non-steroidal anti-inflammatory drugs carry significant side effects resulting in 15,000 deaths per year in the United States.

It is therefore, an object of this invention to provide a penetrating solution of DMSO and safe nutraceuticals, glucosamine and MSM. The goal is to provide rapid penetration through the skin to joints and muscles.

Further and other objects of the invention will be appreciated by those skilled the art.

### SUMMARY OF THE INVENTION

A deeply and rapidly penetrating solution applied topically to deliver a low molecular weight nutraceutical to affected parts of the body.

The solution is comprised of;

(a) 50% dimethyl sulfoxide (DMSO)

(b) 10% methyl-sulfonyl-methane (MSM)

(c) 5% glucosamine

(d) an anti-oxidant to prevent the oxidation of glucosamine in solution

(e) water

Because glucosamine tends to oxidize in gel or lotion form, an anti-oxidant must be added to prolong shelf life.

The acid base ratio (pH) must also be adjusted to prolong glucosamine's shelf life and prevent oxidation. The choice of nutraceuticals over pharmaceuticals, i.e. anti-inflammatories, was not a difficult one. The side effects of the Cox I and Cox II inhibitors are well known and both myself and Dr. Jacob have found a strong reluctance on the part of our patients to use these drugs.

Nutraceuticals such as glucosamine and methyl-sulfonyl-methane (MSM) have been found to be effective by numerous investigators without the side effects of traditional medicines.

### EMBODIMENTS

DMSO gel as a treatment for musculo skeletel disorders including arthritis

DMSO 50%

MSM 10%

Hydroxy Ethyl Cellulose 2.5%

Glucosamine 5%

Citric Acid 1.5%

Sodium Asorbate 0.5%

Purified Water 30%

DMSO lotion as a treatment for musculoskeletel disorders including arthritis

DMSO 50%

MSM 10%

Acetyl Alcohol 5%

Stearyl Alcohol 5%

Glucosamine 5%

Citric Acid 1%

Tocopherol Acetate 1%

Polysorbate 80 1%

Glyceryl mono stearate 2.5.%

Silicone liquid 556 0.5%

The following case histories are offered where the invented solution was employed.

Case 1

Sixty-five year old retired fire chief has had long standing bilateral degenerative disease of knees. Has had total knee replacement on one side. Has been able to avoid surgery on opposite side with equal affliction by using invented solution.

Case 2

Seventy-five year old female who has had long-standing degenerative disease of multiple joints especially knees and hands.

Has had multiple problems with anti-inflammatory drugs of both old Cox I classification and newer Cox II medications.

Has done well on invented solution with no side effects.

Case 3

Physician friend who had shoulder pain secondary to musculo skeletel strain.

Can not take anti-inflammatory drugs.

Took invented solution and was only treatment that allowed him to return to his favorite hobby, golf.

Case 4

Fifty-year old primary care physician with chronic degenerative disease of foot. Invention is the only treatment devoid of side effects that allowed patient to run.

Case 5

Forty-five year old female with chronic lateral epicondylitis of elbow (Tennis elbow). Has had anti-inflammatories with stomach side-effects and not desirous of steroid injections. She gained significant relief with solution of invention.

The big advantage is that the risk benefit ratio is better than any other treatment for musculo skeletel disorders.

Case 6

This invention was used in all triple crown races of the 2002 thoroughbred season. Specific information on request.

Case 7

This invention is part of a lecture given to physicians sponsored by Pfizer Pharmaceuticals. Specific information on request.

We claim:

1. A deep penetrating solution for the topical application of glucosamine, and methyl-solfonyl-methane, the solution comprising:
(a) 50% dimethyl sulfoxide DMSO
(b) 10% methyl-sulfonyl-methane MSM
(c) a polyalcohol to train pleasant moist skin
(d) water
(e) glucosamine
